(19) United States
(12) Patent Application Publication
(10) Pub. No.: US 2025/0018076 A1
(43) Pub. Date: Jan. 16, 2025

(54) METHOD FOR OPERATING A RADIATION EMITTING DEVICE

(71) Applicant: ams-OSRAM Intemational GmbH, Regensburg (DE)

(72) Inventors: Alexander WILM, Regensburg (DE); Clemens HOFMANN, Neutraubling (DE); Johannes HOECHTL, Hagelstadt (DE)

(21) Appl. No.: 18/712,157

(22) PCT Filed: Nov. 10, 2022
(86) PCT No.: PCT/EP2022/081557

§ 371 Date: May 21, 2024

(30) Foreign Application Priority Data
Nov. 30, 2021 (DE) 10 2021 213 500.5

(51) Int. Cl. A61L 2/24; A61L 2/10; A61L 2/26; A61L 9/20; G01V 11/00

(52) U.S. Cl. CPC A61L 2/24; A61L 2/10; A61L 2/26; A61L 9/20; G01V 11/00; A61L 2202/11; A61L 2202/14; A61L 2202/25; A61L 2209/111; A61L 2209/12

(57) ABSTRACT

In at least one embodiment, the method for operating a radiation emitting device, wherein the radiation emitting device is configured to emit radiation into a first part of a room during operation in order to disinfect this first part of the room, comprises a step, in which at least one input signal is provided which is indicative of a change of the irradiation of the room with the radiation from the radiation emitting device. The method further comprises a step in which an output signal for adjusting the operation of the radiation emitting device depending on the input signal is produced.

A method for operating a radiation emitting device is specified. Moreover, a data processing device, a computer program, a computer readable data carrier and a disinfection system are specified.

One object to be achieved is to provide an improved method for operating a radiation emitting device, e.g. a method which allows to automatically recognize and/or react to a potentially dangerous exposure of a human being to the radiation of the radiation emitting device. Further objects to be achieved are to provide a data processing device, a computer program, a computer-readable data carrier and a disinfection system with which such a method can be performed.

These objects are achieved, inter alia, by the subjects of patent claims 1, 11, 12, 13 and 14. Advantageous embodiments and further developments are subject to the dependent claims and can further be extracted from the following description and the figures.

First, the method for operating a radiation emitting device is specified.

According to at least one embodiment, the radiation emitting device is configured to emit radiation into a first part of a room or to irradiate this first part, respectively, during operation in order to disinfect this first part of the room. For example, the radiation emitting device is a UV-radiation emitting device. The radiation emitted by the radiation emitting device may have a radiation maximum, e.g. a global radiation maximum, in the UV-range, e.g. in the UV-C-range. For instance, at least 90% or at least 95% of the emitted radiation or of the radiant flux lies within the UV-range or in the UV-C range. The radiation emitting device may comprise at least one LED. The semiconductor body of the LED may be based on AlGaN.

During performing the method, the radiation emitting device may be arranged at a wall of the room, particularly in the first part of the room. The radiation emitting device may be arranged such that a major part of the radiation emitted by the radiation emitting device, e.g. at least 80% or at least 90% or at least 99% of the radiation, is emitted into the first part of the room.

By illuminating a first part of a room with radiation of the radiation emitting device, particularly with UV-C radiation, this first part can be disinfected. For example, the air in this first part and/or objects in this first part can be disinfected.

The first part of the room may be an upper part of the room. The radiation emitting device may be an upper air disinfection device.

According to at least one embodiment, the method comprises a first step, in which at least one input signal is provided. The first input signal may be indicative of a change of the irradiation of the room with radiation from the radiation emitting device.

The at least one input signal may be a measurement signal or may be representative of a measurement signal generated with help of a detector or a detector system.

The method is, in particular, a computer implemented method. It may be carried out by a data processing device, like a computer, or a system comprising such a data processing device. For providing the input signal, the input signal may be received, e.g. via an interface of the data processing device.

The at least one first signal being indicative of a change of the irradiation of the room particularly means that the at least one input signal comprises information which is an indicator for a change of the irradiation of the room. The information may be suitable for making predictions or assumptions about a change of the irradiation. In other words, the first signal comprises extractable information from which it can be determined or from which an assumption can be made about whether the irradiation of the room has changed and/or to which extent the irradiation of the room has changed. The extractable information can be directly linked to the irradiation, e.g. can be a radiometric measurement, or can be indirectly linked to the irradiation, e.g. can be a measurement of a geometric property, like a distance.

The change of the irradiation of the room may be a change of the distribution of the radiation in the room and/or a change of the intensity of the radiation in the room. The change of the irradiation of the room may be a change of the irradiation of the first part of the room and/or of another part of the room, which is different from the first part. For example, the change of the irradiation of the room is a reduction of the irradiation of a certain region of the room, e.g. due to the appearance of an object between the radiation emitting device and the region shading this region. The change of the irradiation may also be an increased irradiation of a region of a room, e.g. due to the appearance or change of an object reflecting radiation into this region.

According to at least one embodiment, the method comprises a second step, in which an output signal is produced. The output signal may be configured for adjusting the operation of the radiation emitting device. The output signal is produced depending on the input signal.

For example, the output signal is only produced if the input signal indicates a change of the irradiation of the room away from a predetermined irradiation or an undesired change of the irradiation. Additionally, the output signal may only be generated when the change exceeds a predetermined threshold.

For producing the output signal, the input signal or the information extracted from the input signal may firstly be compared to a reference in order to determine if there is a change. Based on this comparison, it may then be determined whether the input signal indicates a critical change, i.e. a change greater than a predetermined threshold, or a non-critical change, i.e. a change below the predetermined threshold. For example, the output signal is produced only in the case of a critical change. Otherwise, the operation of the radiation emitting device may be left as it is.

In at least one embodiment, the method for operating a radiation emitting device, wherein the radiation emitting device is configured to emit radiation into a first part of a room during operation in order to disinfect this first part of the room, comprises a step in which at least one input signal is provided which is indicative of a change of the irradiation of the room with the radiation from the radiation emitting device. The method further comprises a step in which an output signal for adjusting the operation of the radiation emitting device depending on the input signal is produced.

The present invention particularly concerns upper air disinfection. Upper air disinfection involves treating the upper airspace within a room, like a hallway, with radiation, like UV-C radiation, in order to reduce the amount of pathogens, like viruses and bacteria. Since UV-C radiation is also harmful to human beings, the radiation in the part of a room where people are present should be below a certain threshold and below a daily dose, e.g. below 30 J/m².

Looking at the radiation distribution within a room, the room and the objects therein are part of the optical system and any change within the room, e.g. of the objects therein, would affect the irradiation, namely the radiation distribution in the room. Normally, for each configuration, professionals have to ensure that the limits for radiation are fulfilled and the compliance has to be monitored each year to ensure safe operation. If there a person enters the area of radiation, e.g. by standing on a table or changing the properties of the room, e.g. by putting a box on the carport, there is a risk that the person will receive a harmful dose of radiation. Because of the risks of open radiation, upper air disinfection systems are not easily accepted in the market.

The inventors of the present invention had, inter alia, the idea to increase safety by monitoring the irradiated room, e.g. with the help of one or more detectors, in order to recognize a change in the irradiation of the room so that this change can be properly reacted to. This can lead to a safe operation between the maintenance or verification cycles and/or to a reduced amount of maintenance and verification cycles.

According to at least one embodiment, the at least one input signal is indicative of a change of the optical setup of the room. The optical setup of the room comprises, e.g., the spatial arrangement of one or more objects in the room and/or the optical properties of the objects in the room. The walls of the room are herein also understood to be objects in the room. Thus, the at least one input signal may be indicative of a change of the spatial arrangement of at least one object of the room and/or indicative of a change of an optical property of at least one object. The change of optical property may comprise one or more of: a change of a surface of the object, a change of an orientation of the object in the room, a change of the form of the object.

A change of the optical setup of the room may result in a change of the irradiation of the room, e.g. due to changed reflection or absorption. Accordingly, an input signal being indicative of a change in the optical setup may also be indicative of a change of the irradiation. The input signal being indicative of a change of the optical setup of the room may herein be referred to as first input signal.

The first input signal may carry information about the measured actual optical setup of the room. This information may be compared to a reference being representative of a nominal optical setup of the room. If the result of the comparison is that the actual optical setup is different from the nominal optical setup, a corresponding output signal may be generated.

According to at least one embodiment, the at least one input signal is representative of a change of the optical setup in the first part of the room. Particularly, if the optical setup changes, such as the arrangement of objects or the optical properties of objects in the first part of the room, this may result in an increased irradiation of another part of the room (e.g. due to reflection) which was not intended to be irradiated with the radiation of the radiation emitting device. Such an increase of the irradiation may be dangerous for a person in the other part of the room.

According to a further embodiment, the change of optical setup is the appearance of an object in the first part of the room. For example, the change in the optical setup is an appearance of a person in the first part of the room or of a portion of the person in the first part of the room. In order to detect such an appearance, a safety radiation barrier or radiation curtain may be used between the first part of the room and one or more other parts of the room.

According to a further embodiment, the change of the optical setup is the change of an object in the first part of the room and/or change in the position of the object inside the first part of the room. The change of the object may be a change of the optical properties of the object, e.g. a change of reflectivity, for the radiation of the radiation emitting device.

According to a further embodiment, the at least one input signal is indicative of a change of the irradiation of a second part of the room with the radiation of the radiation emitting device. The second part of the room is different from the first part of the room.

For example, the at least one input signal carries information about a measured irradiance of a certain area in the second part of the room with the radiation of the radiation emitting device. The certain area may be a detection area of a detector, e.g. at a wall of the room in the second part. This information may be compared to a reference irradiance. The reference irradiance may be a maximum allowed irradiance. If, based on this comparison, it is determined that the measured irradiance exceeds the reference irradiance, e.g. by more than a predetermined threshold, a corresponding output signal may be produced. The input signal being indicative of a change of the irradiation of the second part of the room may herein be referred to as second input signal.

According to at least one embodiment, the first part is an upper part of the room and the second part is a lower part of the room. For example, the whole room is divided into the first part and the second part. The second part may reach from the bottom of the room to a certain height and the first part may reach from this certain height to the ceiling of the room. The certain height may be at least ½ or at least ¾ of the total height of the room.

According to at least one embodiment, the at least one input signal, e.g. the second input signal, is a signal produced with help of at least one radiation detector. The radiation detector is configured to detect an exposure to the radiation of the radiation emitting device. For instance, the radiation detector is configured to detect the radiation and/or to measure the irradiance of the radiation from the radiation emitting device.

During operation, the radiation detector may be arranged at a wall of the room, particularly in the second part of the room. The radiation detector may be a UV-radiation detector.

According to at least one embodiment, the at least one input signal, e.g. the first input signal, is a signal produced with the help of at least one of: a presence detector, a camera, an IR-camera, a LIDAR system, a radar system, a structured light system, a ToF system, like a 3D-ToF system or a 1D-TOF system.

The mentioned detectors or detector systems, respectively, are particularly suited for measuring the optical setup of the room, particularly the spatial arrangement of the objects in the room or the position of one or more objects in the room.

For example, one or more of the above-mentioned detectors or detector systems are used to produce the above-mentioned safety radiation barrier or radiation curtain.

According to a further embodiment, the output signal is configured to cause an automatic adjustment of the operation of the radiation emitting device. Additionally or alternatively, the output signal is configured to call for an adjustment of the operation of the radiation emitting device. For example, the output signal may be configured to automatically switch off the radiation emitting device or to automatically adjust the operation of the radiation emitting device such that the radiation emitting device emits less or more radiation, e.g. a smaller or larger radiant flux.

Additionally or alternatively, the output signal may be configured to indicate to a user of the radiation emitting device that he/she should manually switch off the radiation emitting device or that he/she should manually reduce the radiant flux emitted by the radiation emitting device. For example, the output signal is configured to visually and/or acoustically indicate to a user that he/she should change the operation of the radiation emitting device. For example, the output signal is configured to produce a visual and/or acoustic alert.

According to at least one embodiment, the at least one input signal is indicative of a change of the irradiation of the first part of the room by radiation of the radiation emitting device.

For example, the at least one input signal carries information about a measured irradiance in a certain area of the first part of the room with the radiation of the radiation emitting device. The certain area may be an area at a wall of the room in the first part of the room, e.g. a detection area of a detector. This information may be compared to a reference irradiance. The reference irradiance may be a minimum required irradiance in order to have sufficient disinfection. If, based on this comparison, it is determined that the measured irradiance falls below the reference irradiance, e.g. by more than a predetermined threshold, a corresponding output signal may be produced. The input signal being representative of a change of the irradiation of the first part of the room may herein be referred to as third input signal.

The output signal produced depending on the third input signal may be configured to automatically adjust the operation of the radiation emitting device such that the radiant flux emitted by the radiation emitting device is increased. In this way aging effects, like aging of the used LED or of the used optics, or electronic variations can be compensated.

According to at least one embodiment, the third input signal is a signal produced with the help of at least one radiation detector. The radiation detector is, e.g., configured to measure the irradiance of the radiation from the radiation emitting device. During operation, the radiation sensor may be arranged in the first part of the room, e.g. at a wall of the room. The radiation detector may be a UV-radiation detector. The radiation detector may be arranged at the radiation emitting device, e.g. in the radiation path, and may detect a small portion of the emitted radiation.

Next, the data processing device is specified. The data processing device may be a computer. The data processing device comprises a processor configured to perform the method for operating a radiation emitting device disclosed herein.

Next, the computer program and the computer-readable data carrier are specified. The computer program and the computer-readable data carrier comprise instructions which, when executed by a computer, cause the computer to carry out the method for operating a radiation emitting device disclosed herein.

Next, the disinfection system is specified. The disinfection system may, particularly, be used to carry out the method for operating a radiation emitting device disclosed herein. All features disclosed for the method are therefore also disclosed for the system and vice versa. The disinfection system is, e.g., an upper air disinfection system.

According to at least one embodiment, the system comprises a radiation emitting device for irradiating a first part of a room with radiation in order to disinfect this first part of the room.

According to at least one embodiment, the disinfection system comprises at least one detector for detecting a physical quantity, wherein a change of the physical quantity is indicative of a change of the irradiation of the room with the radiation of the radiation emitting device. For example, the disinfection system comprises one or more of the above-mentioned detectors or detector systems.

According to at least one embodiment, the at least one detector is configured to generate a first input signal comprising information about the detected physical quantity.

According to at least one embodiment, the disinfection system is configured to perform the method disclosed herein.

According to at least one embodiment, the disinfection system comprises the data processing device disclosed herein.

The input signals from the one or more detectors or detector systems can be transmitted to the data processing device via a wired connection or wirelessly. The data processing device may be incorporated into the at least one detector (system) or into the radiation emitting device. Alternatively, the data processing device may be a separate or external device. The output signal may be transmitted to the radiation emitting device or to a control unit for controlling the radiation emitting device via a wired connection or wirelessly. Accordingly, the data processing device, the radiation emitting device and/or the detector(s) may comprise appropriate interfaces for the wired or wireless connection. The wireless connection may be Bluetooth, Wifi, Lifi or a radio connection.

According to at least one embodiment, the disinfection system is configured to generate a radiation curtain between the first part of the room and a further part of the room in order to detect when an object is passing through the radiation curtain into the first part of the room. For this purpose, the disinfection system may comprise a detector system with a further radiation source, e.g. a laser, in order to produce the radiation curtain, i.e. a radiation barrier. A detector of this detector system may be configured to measure the radiation of the further radiation source so that a change in the measured radiation may indicate an object passing through the radiation curtain. The detector system for realizing the radiation curtain or barrier, respectively, may be a LIDAR system or a 1D ToF system or a radar system.

Hereinafter, the method for operating a radiation emitting device, the data processing device and the disinfection system described herein are explained in more detail with reference to drawings on the basis of exemplary embodiments. Identical reference signs indicate similar, similar acting or identical elements in the individual figures. It is to be understood that the embodiments shown in the figures are illustrative representations and are not necessarily drawn to scale. Rather individual elements may be shown exaggeratedly large for better understanding. Insofar as elements or components in the various figures correspond in their function, their description is not repeated for each of the following figures. For reasons of clarity, elements may not be given corresponding reference signs in all figures.

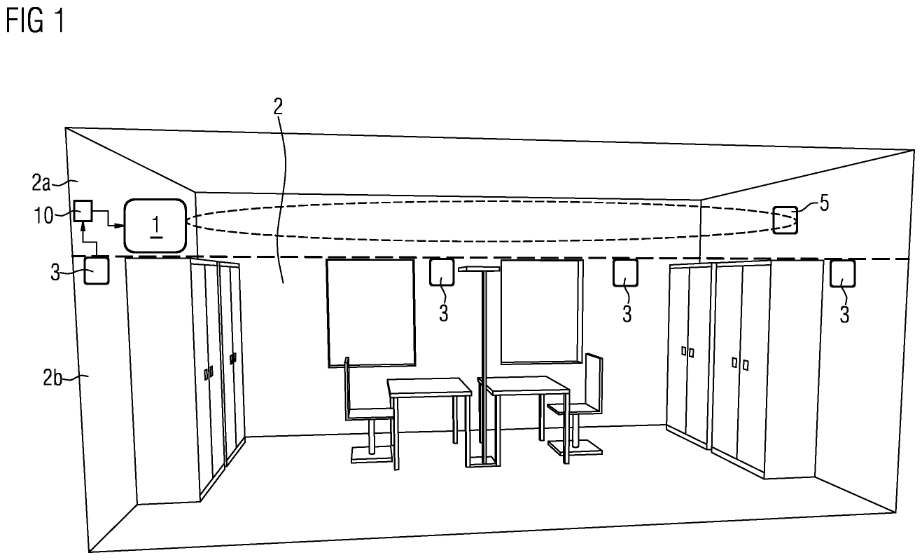

FIGS. 1 to 3 show different exemplary embodiments of the disinfection system, and

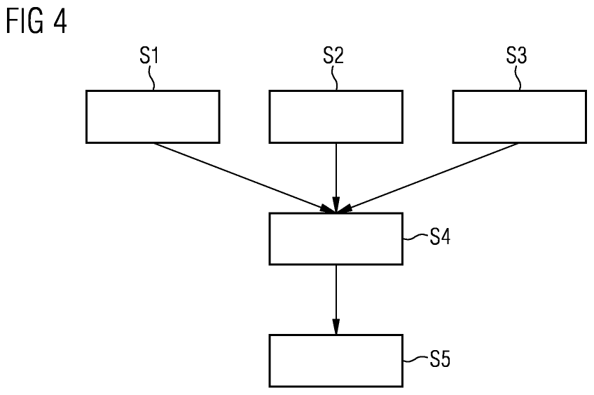

FIG. 4 shows a flowchart of an exemplary embodiment of the method for operating the radiation emitting device.

FIG. 1 shows a first exemplary embodiment of the disinfection system. The disinfection system comprises a radiation emitting device 1, a data processing device 10 and radiation detectors 3, 5. The disinfection system is arranged in a room 2.

The radiation emitting device 1 is positioned at a wall of the room 2 and is configured to emit radiation which disinfects the objects it hits or the medium which it traverses. For example, the radiation emitted by the radiation emitting device 1 is UV-C radiation, e.g. radiation with an intensity maximum in the UV-C range.

The radiation emitting device 1 irradiates a first part 2a of a room 2 with its radiation (indicated by the dashed ellipse). The first part 2a is an upper part of the room 2, reaching from the ceiling of the room 2 to a certain height of the room 2. For example, the radiation emitting device 1 emits a major part or all of its radiation into the first part 2a of the room 1. For example, at least 95% of the radiation emitted by the radiation emitting device 1 is emitted into the first part 2a.

The room 2 comprises a second part 2b which, in this case, is a lower part of the room 2. The second part 2b reaches from the bottom of the room 2 to the first part 2a. The virtual border between the two parts 2a, 2b is indicated by the dashed line. As can be seen in FIG. 1, no or almost no radiation of the radiation emitting device 1 is emitted into the second part 2b. For example, during intended operation of the disinfection system, at most 5% of the radiation emitted by the radiation emitting device 1 enters into the second part 2b.

The radiation detectors 3 are arranged in the second part 2b of the room 2 at the wall of the room 2. The radiation detectors 3 are configured to detect the radiation emitted by the radiation emitting device 1. For example, the detectors 3 are configured to measure the irradiance of the radiation of the radiation emitting device 1 hitting a respective detection area or the detector.

The radiation detector 5 is also configured to detect the radiation emitted by the radiation emitting device 1, e.g. to measure the irradiance of the radiation hitting a detection area. The radiation detector 5 is arranged in the first part 2a of the room 2.

The radiation detectors 3 are each configured to produce an input signal (herein also called second input signal) which is indicative of the measured irradiance. Thus, the input signals of the detectors 3 are indicative of the irradiation of the second part 2b of the room 2 with radiation of the radiation emitting device 1.

The second input signals of the radiation detectors 3 are transmitted to the data processing device 10. In the present case, the transmission is a wired transmission.

Alternatively, a wireless transmission may be used, e.g. via Bluetooth or Wifi.

The data processing device 10 is configured to produce an output signal depending on these second input signals. For example, the data processing unit 10 is configured to compare information carried by the second input signals to a reference. The reference may represent a maximum allowed measured irradiance. If the result of the comparison is that the reference is exceeded, the data processing unit 10 may produce a corresponding output signal. This output signal 10 is then transmitted to the radiation emitting device 1 or a control unit of the radiation emitting device 1. Based on this output signal, the radiation emitting device 1 is automatically switched off or the radiant flux emitted by the radiation emitting device 1 is reduced automatically.

In the intended use of disinfection system, the maximum allowed irradiance measured with the detectors 3 is not exceeded or at least not exceeded by more than a predetermined threshold. Accordingly, the radiation emitting device 1 can be operated such that a sufficient high radiant flux is emitted in order to disinfect the air in the first part 2a of the room 2. However, due to an unforeseen or undesired change in the room 2, the radiation emitted into the second part 2b of the room 2 may increase such that it could be harmful for a person in the room 2. Such an unforeseen or undesired change may be, e.g., that an object is placed in the first part 2a of the room which reflects radiation of the radiation emitting device 1 into the second part 2b of the room. For instance, a box could be put on one of the cupboards. With the disinfection system described herein, an automatic recognition and reaction to such a change can be realized which helps to protect a person in the room 2.

In order to guarantee that the first part 2a of the room is sufficiently disinfected by the radiation of the radiation emitting device 1, the radiation detector 5 is used. The detector 5 produces an input signal (herein also called first input signal) which is representative of the irradiation of the first part 2a of the room 2 by radiation from the radiation emitting device 1. The third input signal is transmitted to the data processing device 10, e.g. via wired connection or wireless connection. The data processing device 10 may be configured to compare information carried by the third input signal with a reference. The reference may be representative of a minimum measured irradiance which is necessary for a sufficient disinfection. If the result of the comparison is that the reference is undershot, e.g. by more than a certain threshold, the data processing device 10 may produce a corresponding output signal configured to automatically operate the radiation emitting device 1 such that the radiant flux is increased. In this way a sufficient disinfection of the first part 2a of the room 2 can be obtained.

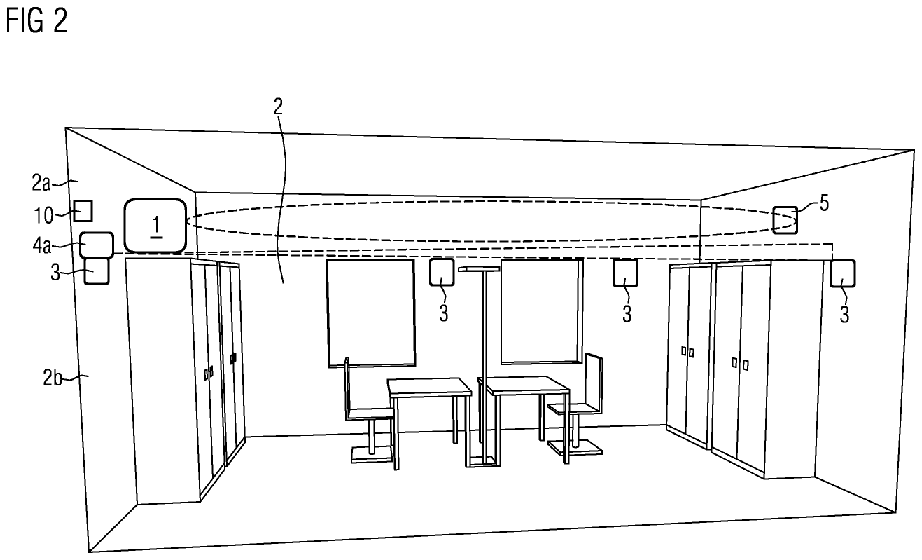

FIG. 2 shows a second exemplary embodiment of the disinfection system. The disinfection system works, in principle, in the same way as the disinfection system of FIG. 1. However, in the second exemplary embodiment, the disinfection system is additionally configured to produce a radiation curtain between the first part 2a of the room 2 and the second part 2b of the room 2. The radiation curtain is indicated by the dashed cone.

The radiation curtain is used to detect when an object, like the head of a person, enters from the second part 2b into the first part 2a. For this purpose, the disinfection system comprises a detection system with a radiation detector 4a and a further radiation source, e.g. a laser source. The further radiation source is here incorporated into the radiation detector 4a. Both are arranged at a wall of the room 2. The radiation emitted by the further radiation source defines the radiation curtain. The radiation reflected from the opposite wall, e.g. via a mirror of the detector system, is measured by the radiation detector 4a. If the detected radiation changes, this might be an indication for an object passing through the radiation curtain. The detection system used to realize the radiation curtain may be a LIDAR system or a radar system or a 1D-TOF system.

The detector 4a produces an input signal which is indicative of the measured radiation of the further radiation source. This input signal is herein also referred to as first input signal. This first input signal is transferred to the data processing device 10. The data processing device 10 may then produce a corresponding output signal based on this first input signal, e.g. when it is determined that the first input signal indicates an object in the radiation curtain. The output signal is then transmitted to the radiation emitting device 1 or a control unit thereof, e.g. in order to automatically switch off the radiation emitting device 1 or in order to automatically reduce the radiant flux emitted by the radiation emitting device 1.

Thus, in the second exemplary embodiment of FIG. 2, three different input signals can be transmitted to the data processing unit 10 in order to produce one or more output signals for adjusting an operation of the radiation emitting device 1.

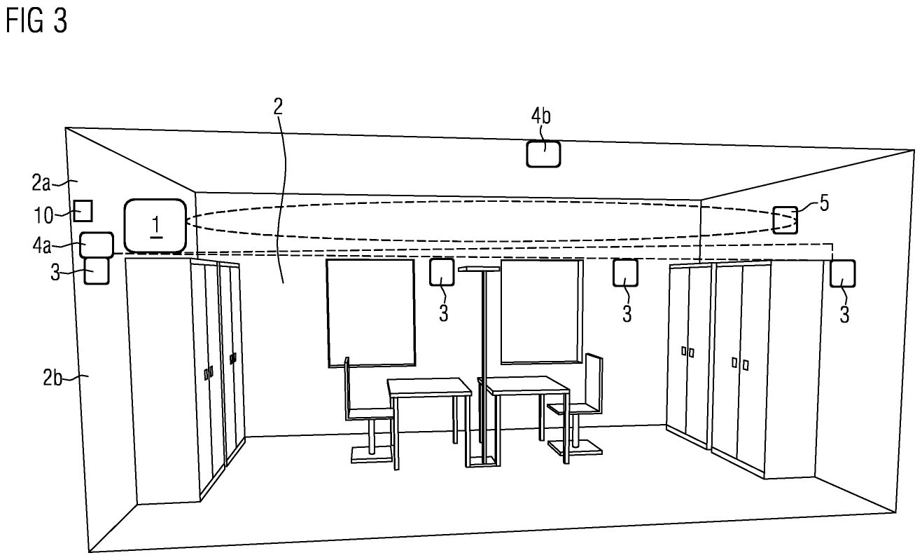

In the exemplary embodiment of FIG. 3, a further detector 4b is used. The further detector 4b is, e.g., configured to measure a change in the optical setup of the first part 2a of the room 2. The detector 4b may be a presence detector/sensor or a camera or a camera of a camera system, like an IR camera. The camera system may comprise an additional radiation source, like an IR radiation source, for compensating for changes due to daylight. The detector 4b may also be part of a LIDAR system or of a radar system or of a structured light system or of a 3D-ToF system.

With the help of detector 4b, the first part 2a is observed and a change of the spatial arrangement of the objects in the first part 2a of the room 2 or the appearance of an additional object in the first part 2a of the room may be detected. The produced first input signal of the detector 4b may then be transmitted to the data processing device 10.

Based on this first input signal, a corresponding output signal may be produced for adjusting the operation of the radiation emitting device 1.

In the previous exemplary embodiments, the output signal of the data processing device 10 was used to automatically adjust the operation of the radiation emitting device 1, e.g. by turning off the radiation emitting device 1 or reducing or increasing the radiant flux of the radiation emitting device 1. However, the output signal of the data processing device 10 could also be used to call for an adjustment of the operation of the radiation emitting device 1, e.g. by producing an alert.

FIG. 4 shows a flowchart of an exemplary embodiment of the method for operating a radiation emitting device. This method may be used for operating the radiation emitting device 1 of one of the previous figures, e.g. of that of FIG. 3. In steps S1, S2 and S3, different input signals are received which are each indicative of a change of the irradiation of the room 2 with the radiation of the radiation emitting device 1.

For example, in step S1 a first input signal is received by the data processing device 10 which is indicative of measurements of the detectors 4a, 4b. This first input signal is indicative of a change of the optical setup in the first part 2a of the room 2, e.g. for the appearance of an additional object which has not been there before.

In step S2, a second input signal is received by the data processing device 10. The second input signal is indicative of a measurement of at least one of the detectors 3. The second input signal is indicative of a change, like an increase, of the irradiation in the second part 2b of the room 2 with radiation of the radiation emitting device 1. This increase may be a consequence of the new object in the first part 2a reflecting radiation into the second part 2b.

In step S3, a third input signal is received by the data processing device 10, which is indicative of a measurement of detector 5. The third input signal is, e.g., indicative of a change, like a reduction, of the irradiation in the first part 2a of the room 2 with radiation of the radiation emitting device 1. The reduction may be due to the object partially shading the detector 5.

In step S4, the data processing unit 10 produces an output signal based on one or more of the input signals received in steps S1, S2 and S3. The output signal is configured for adjusting the operation of the radiation emitting device 1. For example, the output signal is configured to automatically adjust the operation of the radiation emitting device 1 or to call for an adjustment of the operation of the radiation emitting device 1. In this case, the output signal may be configured to turn off the radiation emitting device 1, since the increase in irradiation in the second part 2b may be dangerous for a person.

In step S5, the operation of the radiation emitting device 1 is adjusted based on the output signal. For example, the operation of the radiation emitting device 1 is automatically adjusted with the help of the output signal or the output signal is used to call for an adjustment and an operator of the disinfection system then manually adjusts the operation of the radiation emitting device 1.

The invention described herein is not limited by the description in conjunction with the exemplary embodiments. Rather, the invention comprises any new feature as well as any combination of features, particularly including any combination of features in the patent claims, even if said feature or said combination per se is not explicitly stated in the patent claims or exemplary embodiments.

### REFERENCES

1 radiation emitting device
2 room
2a first part of the room
2b second part of the room
3 detector
4a, 4b detector
5 detector
10 data processing device
S1 to S5 method steps

1. A method for operating a radiation emitting device, wherein
the radiation emitting device is configured to emit radiation into a first part of a room during operation in order to disinfect this first part of the room, wherein the whole room is divided into the first part and a second part and the first part is an upper part of the room and the second part is a lower part of the room, wherein
a radiation curtain is generated between the first part of the room and the second part of the room so as to detect when an object is passing through the radiation curtain from the second part into the first part of the room, the radiation curtain being defined by radiation emitted by a further radiation source, wherein
the method comprises
providing at least one input signal which is indicative of a change of the irradiation of the room with the radiation from the radiation emitting device, the input signal comprising a first input signal indicative of the measured radiation of the further radiation source,
producing an output signal for adjusting the operation of the radiation emitting device depending on the input signal.

2. The method according to claim 1, wherein
the at least one input signal is indicative of a change of the optical setup of the room.

3. The method according to claim 2, wherein
the change of the optical setup is the appearance of an object in the first part of the room.

4. The method according to claim 1, wherein
the change of the optical setup is the change of an object in the first part and/or a change in the position of the object.

5. The method according to claim 1, wherein
the at least one input signal is indicative of a change in the irradiation of a second part of the room with the radiation of the radiation emitting device, the second part being different from the first part.

6. The method according to claim 5, wherein
the at least one input signal is a signal produced with the help of at least one radiation detector configured to detect an exposure to the radiation of the radiation emitting device.

7. The method according to claim 1, wherein
the at least one input signal is a signal produced with the help of at least one of: a presence detector, a camera, an IR-camera, a LIDAR system, a radar system, a structured light system, a 3D-ToF system, a 1D-TOF system.

8. The method according to claim 1, wherein
the output signal is configured to cause an automatic adjustment or to call for an adjustment of the operation of the radiation emitting device.

9. The method according to claim 1, wherein
the at least one input signal is indicative of a change of the irradiation of the first part of the room with the radiation of the radiation emitting device.

10. The data processing device comprising a processor configured to perform the method of claim 1.

11. A computer program comprising instructions which, when the program is executed by a computer, cause the computer to carry out the method of claim 1.

12. A computer-readable data carrier comprising instructions which, when executed by a computer, cause the computer to carry out the method of claim 1.

13. A disinfection system comprising
a radiation emitting device for irradiating a first part of a room with radiation in order to disinfect this first part of the room,
at least one detector for detecting a physical quantity, a change of the physical quantity being indicative of a change of the irradiation of the room with the radiation of the radiation emitting device, and
a further radiation source, configured to generate a radiation curtain between the first part of the room and a further part of the room in order to detect when an object is passing through the radiation curtain into the first part of the room, wherein
the at least one detector is configured to generate a first input signal comprising information about the detected physical quantity,
the disinfection system is configured to perform the method according to claim 1.

14. (canceled)
